(19) United States
(12) Patent Application Publication
(10) Pub. No.: US 2003/0003503 A1
(43) Pub. Date: Jan. 2, 2003

(54) Enhancing sensitivity and equimolar detection through modifications of the reaction environment

(76) Inventors: Tenlin S. Tsai, Mountain View, CA (US); Wesley K. Tamashiro, Yorba Linda, CA (US); Cheng F. Yan, Irvine, CA (US); James D. Barr, Tustin, CA (US)

Correspondence Address: BECKMAN COULTER INC, 4300 NORTH HARBOR BOULEVARD, P O BOX 3100, FULLERTON, CA, 928343100

(21) Appl. No.: 09/896,046

(22) Filed: Jun. 29, 2001

(51) Int. Cl. G01N 33/53; G01N 33/537; G01N 33/543; C12N 9/99

(52) U.S. Cl. 435/7.1; 435/184

(57) ABSTRACT

This invention provides methods of improving assays for the quantitation of a protein in a sample by enhancing equimolar detection of the protein. Detection is enhanced by modifying the reaction environment of the protein. This invention further provides improved reagent particles for use in the detection and/or quantitation of an analyte in a sample, where the reagent particles comprise two or more coatings of the same or a different substance, such as an antibody, immobilized on an insoluble particle. This invention further provides a method of increasing the sensitivity of particle-enhanced immunoassays by increasing the size of the aggregates that are formed in the assay. Aggregate size is increased by combining the sample with three different antibodies: an analyte-specific antibody immobilized on an insoluble particle, a second antibody specific analyte, and a linker antibody which specifically binds the second antibody but does not bind to the analyte.

### FIELD OF THE INVENTION

This invention relates to the field of diagnostics, and more particularly to assays having improved equimolar detection or quantitation of a protein of interest in a sample, wherein the sample may comprise both the unbound and complexed forms of the protein.

### BACKGROUND OF THE INVENTION

Proteins are composed of long chains of amino acids. The structure of proteins can be considered on four different levels. The primary structure refers to the specific order of amino acids in the polymer chain. The secondary structure refers to the interactions among and between the amino acids and their side chains and groups (such as hydrogen bonding and disulfide bridge) to form such structures as alpha helices and beta pleated sheets. The tertiary structure refers to the three-dimensional structure of the protein, which is also referred to as a protein's conformation. The quaternary structure refers to the spatial arrangement of individual polypeptides or “subunits” of multisubunit proteins.

The native conformation of a protein is only marginally stable. Thus, many proteins which are removed from their native environment and subsequently purified undergo conformational changes which can cause a loss of biological activity, such as enzyme activity or antibody-binding capacity. In particular, the individual subunits of multisubunit protein complexes may undergo dramatic conformational changes when separated from the other subunits of the complex and stored in a liquid medium. Since a protein's biological activity depends on its conformation, stability of a protein can be measured as a function of the protein's biological activity.

Protein subunits of multisubunit protein complexes can exist in various forms in a test sample, including unbound forms and different types of complexed forms. Antibodies can be prepared according to methods known to those of ordinary skill in the art that specifically bind to a particular epitope on a protein subunit of interest. However, under conventional immunoassay reaction conditions for measuring total protein subunit concentration in a sample, the conformations of the subunits in various complexed forms may be such that the antibody is prevented from binding to the epitope on these complexed subunits. As a result, conventional immunoassays do not accurately measure the total amount of the various forms of the protein in a sample. Accurate total protein subunit measurements are critical in clinical settings in order to properly diagnose and treat various disorders. The lack of accuracy in conventional assays results in discordant measurements, both between different assays and within the same assay performed on the same instrument at different times.

Troponin is a generic term used to identify a multisubunit muscle protein integrally involved in the calcium-dependent regulation of muscle contraction. Troponin exists in both cardiac and skeletal muscle as a non-covalently bound complex of three subunits: troponin T (tropomyosin binding subunit), troponin C (calcium binding subunit), and troponin I (ATPase inhibitory subunit). These troponin subunits exist as various tissue-specific isoforms. Troponin C exists as two isoforms, one from cardiac and slow-twitch muscle and one from fast-twitch muscle. Troponin I and troponin T are expressed as different isoforms in slow-twitch: fast-twitch and cardiac muscle (Biochem. J., 171:251-259 (1978); J. Biol. Chem., 265:21247-21253 (1990); Hum. Genet., 88:101-104 (1991); Circul. Res., 69:1226-1233 (1991)). The unique cardiac isoforms of troponin I (cTnI) and troponin T (cTnT) allow them to be distinguished immunologically from the other troponin isoforms from skeletal muscle. Therefore, the release into the blood of troponin I and T from a damaged heart muscle has been related to cases of unstable angina and acute myocardial infarction (AMI).

After myocardial infarction, cardiac troponin T (cTnT) levels increase and remain elevated for an extended period. However, it has been reported that in a variety of disease states, cTnT is also expressed in skeletal muscle, which contributes to a lack of cardiospecificity of this protein. Furthermore, uremia, a condition associated with cardiomyopathy, is associated with elevated cTnT. Thus, a lack of absolute cardiospecificity makes this marker less than optimal for use in the early diagnosis of acute myocardial infarction (AMI).

Cardiac troponin I (cTnI) is also released after acute myocardial infarction. In contrast to cTnT, cTnI has never been found in a healthy population, which includes marathon runners, in people with skeletal disease, or in patients undergoing non-cardiac operations. Thus, cTnI is a more specific marker for the diagnosis of AMI than other serum proteins.

Troponin I, T, and C can exist in the unbound forms, as binary complexes (I/T, I/C, and T/C) and as a ternary complex (I/T/C). Since the conformations of troponin I, T and C change upon binding when forming binary and ternary complexes (Biochemistry, 33:12800-12806 (1994); J. Biol. Chem., 254:350-355 (1979); Am. Rev. Biophys. Biophys. Chem.: 16:535-559 (1987)), an understanding of the conformational changes of troponin I and troponin T and the heterogeneity of the proteins in the blood is critical for the development of accurate diagnostic procedures for measuring troponin I and troponin T concentrations.

A variety of immunoassays have been developed utilizing antibodies that can distinguish between the three troponins, and also between their different isoforms. Most immunoassays are designed to determine the concentration of a given marker (e.g., cTnI or cTnT) in a patient's serum by comparing immunoassay results with the patient's serum to those obtained with control reagents of known concentration. Immunoassays have also been described which are specific for cTnT. See, for example, Katus, et al., Circulation, 83(3):902-912 (1991). An immunoassay for cTnT is also commercially available from the Boehringer Mannheim Corporation, Indianapolis, Indiana. U.S. Pat. No. 6,174,686, issued to Buechler et al., discloses stabilized compositions of troponin and assay systems and specialized antibodies for the detection and quantitation of troponin I and troponin T in body fluids. However, the currently available cTnI assays often produce differing results. One limitation in the development of immunoassays for the troponin subunits involves the instability of the subunits, whether in the free (unbound) or complexed state.

Another important reason that the assays may differ in their responses is due to the various complexed forms of cTnI present in circulation or in biochemical preparations. In addition to “free” cTnI and its binary and ternary complexes with cTnC and cTnT (e.g., I/C, I/T, T/C, or I/C/T), cTnI may exist in phosphorylated, oxidized, and proteolytically degraded forms. The latter modified forms of cTnI also may exist as binary or ternary complexes. All of these forms may have different recognition patterns in different immunoassays. (P. Datta, et al., Clin. Chem., 45:2266-2269 (1999); J. Neumann, et al., J. Cardiovascular Pharmacology, 33:157-162 (1999); H. Nagasaki, et al., Clin. Chem., 45(4): 486-496 (1999); G. S. Bodor, et al., Circulation, 96(5):1495-1500 (1997); J. P. Chapelle, Clin. Chem. Lab. Med., 37(1):11-20 (1999); E. Al-Hillawi, et al., Eur. J. Biochem., 256:535-540 (1998); S. U. Reiffert, et al., Biochemistry, 37:13516-13525 (1998); A. S. Jaffe, Clinica Chiimica Acta, 284:197-211 (1999); J. L. Bock, Am. J. Clin. Pathol., 112:739-741 (1999)). For example, the predominant form of cTnI in acute myocardial infarction patients is the binary complex I/C. Samples treated with a calcium ion (Ca²⁺) chelator such as EDTA would contain mostly free cTnI because the chelation of Ca²⁺ disrupts I/C and I/C/T complexes.

A number of instruments have been designed and are commercially available to measure total cTnI in the blood of suspected heart attack victims. These include the Abbott AxSYM®, the Dade OPUS®, the Bayer IMMUNO-1®, the Beckman ACCESS®, and the Dade STRATUS®. Each of these instruments measures cTnI by reacting different cTnI epitopes with different antibodies. One assay, for example, employs an antibody that binds an epitope near the C-terminus of the molecule, whereas another assay employs an antibody that binds an epitope near the middle of the molecule or a the N-terminus.

The epitope measured may be another source of inaccuracy and/or lower sensitivity of the assay. This is due to the fact that the epitopes in some of the complexed forms of cTnI in the test sample may be inaccessible to the antibody. Consequently, the assay is not able to accurately measure total cTnI concentration. Therefore, depending on the particular complexed form of the cTnI, the same assay method can produce varying measurements of total cTnI. These variations can be critical in accurately diagnosing a medical condition such as stratification and risk assessment of AMI in a patient.

Therefore, there is still a need for immunoassays having improved sensitivity and improved equimolar detection of proteins of samples, including the detection and quantitation of the total amount of a protein subunit in a sample.

### SUMMARY OF THE INVENTION

The present invention provides improved assays for the detection and/or quantitation of an analyte in a sample. More specifically, one aspect of this invention provides an improved assay for the detection and/or quantitation of a protein of interest in a sample, wherein the sample may contain various forms of the protein. The assay of this invention provides improved equimolar detection of the total amount of a protein in a sample, and therefore enhances the sensitivity of protein detection relative to conventional assays.

Accordingly, one aspect of this invention provides an improved particle-enhanced assay for the quantitation of the total amount of a protein in a sample, wherein the sensitivity and equimolar detection of the protein is enhanced by modifying the reaction environment of the sample. This modification causes the epitope on all forms (i.e., unbound and complexed) of the protein of interest to be accessible to its specific binding partner, thereby allowing the binding partner to have equal access to its epitope on all forms of the protein in the sample. More specifically, one embodiment of this invention provides an assay for determining the total amount of a protein in a sample, comprising:

a) providing a sample containing an unknown amount of the protein, wherein the sample may comprise both unbound and complexed forms of the protein;

b) providing reagent particles comprising insoluble particles having immobilized thereon a binding partner specific for an epitope of the protein;

c) forming a reaction mixture by combining the sample, the reagent particles, and a factor, wherein the factor causes conformational changes in the unbound and/or complexed forms of the protein to allow the epitope on all forms of the protein to be accessible to the binding partner;

d) incubating the reaction mixture under conditions that allow binding between the immobilized binding partner and the epitope, wherein the binding results in aggregation of the particles; and

e) determining the amount of total protein in the sample.

Another aspect of this invention comprises an improved particle-enhanced assay for the detection and/or quantitation of an analyte in a sample, wherein the assay utilizes improved reagent particles having two or more coatings immobilized thereon. More specifically, one aspect of this invention provides improved reagent particles comprising an insoluble particle, an inner coating (e.g., a protein or antibody) immobilized on the surface of the particle, and an outer coating of binding partner that is specific for the analyte of interest and which is immobilized on the inner coating. The double coating on the insoluble particles enhances the specific binding and epitope recognition between the analyte-specific binding partner and the analyte of interest in a particle-enhanced assay.

Yet another aspect of this invention provides an improved particle-enhanced immunoassay for the detection and/or quantitation of an analyte in a sample, wherein the sensitivity of the immunoassay is enhanced by increasing the extent of particle aggregation and increasing the size of the aggregates that are formed in the assay. More specifically, one embodiment of this invention provides an assay for determining the amount of an analyte of interest in a sample, comprising:

a) providing:

i) a sample containing an unknown amount of the analyte;

ii) reagent particles comprising insoluble particles having immobilized thereon a first antibody that is immunoreactive with the analyte;

iii) a second antibody that is immunoreactive with the analyte, and

iv) a linker antibody which specifically binds the second antibody but does not bind to the analyte;

b) forming a reaction mixture by combining the sample, the reagent particles, the second antibody, and the linker antibody;

c) incubating the reaction mixture under conditions that allow binding between the immobilized first antibody and the analyte, binding between the second antibody and the analyte, and binding between the second antibody and the linker antibody, wherein the bindings result in aggregation of the particles; and

d) determining the amount of analyte in the sample.

Additional objects, advantages and novel features of this invention shall be set forth in part in the description that follows, and in part will become apparent to those skilled in the art upon examination of the following specification or may be learned by the practice of the invention. The objects and advantages of the invention may be realized and attained by means of the instrumentalities, combinations, and methods particularly pointed out in the appended claims.

### BRIEF DESCRIPTION OF THE FIGURES

The accompanying drawings, which are incorporated herein and form a part of the specification, illustrate preferred embodiments of the present invention and, together with the description, serve to explain the principles of the invention.

In the Figures:

FIG. 1 is an illustration of the random orientation of antibodies immobilized on a conventional reagent particle used in particle-enhanced immunoassays.

FIG. 2 is an illustration of one embodiment of an improved reagent particle of this invention having an inner coating and an outer coating comprising an antibody that is immunoreactive with an analyte of interest.

FIG. 3 is an illustration of particle aggregation that occurs in particle-enhanced assays, comparing particle aggregation in conventional assays (area inside triangle) and the improved particle aggregation (area inside dashed square) utilizing three different antibodies according to a method of this invention.

FIG. 4 is a graph showing the detection and separation of different levels of cTnI in a particle-enhanced assay in which a factor that modifies the reaction environment was not added to the assay reaction mixture.

FIG. 5 is a graph showing the detection and separation of different levels of cTnI in a particle-enhanced assay in which a factor that modifies the reaction environment was added to the assay reaction mixture.

### DETAILED DESCRIPTION OF THE INVENTION

One embodiment of this invention provides improved assays for the detection and/or quantitation of the total amount of a protein of interest in a sample by improving the equimolar recognition of a binding partner to a specific epitope on all forms of the protein. The improved assays of this invention overcome this shortcoming of conventional assays by modifying the reaction environment of the protein, thereby exposing the specific epitope on all forms of the protein to the binding partner. As a result, a binding partner specific for that epitope has equal access to the epitope in all forms of the protein, which improves the accuracy in the measurement of a protein in a sample.

More specifically, one embodiment of this invention for improving equimolar recognition of a protein of interest in a sample comprises:

a) providing a sample containing an unknown amount of the protein, wherein the sample may comprise both unbound and complexed forms of the protein;

b) providing reagent particles comprising insoluble particles having immobilized thereon a binding partner specific for an epitope of the protein;

c) forming a reaction mixture by combining the sample, the reagent particles, and a factor, wherein the factor causes conformational changes in the unbound and/or complexed forms of the protein to allow the epitope on all forms of the protein to be accessible to the binding partner;

d) incubating the reaction mixture under conditions that allow binding between the immobilized binding partner and the epitope, wherein the binding results in aggregation of the particles; and

e) determining the total amount of the protein in the sample.

As used herein, the term “protein” refers to a polymer of amino acids chemically bound by amide linkages (CONH), and includes both unbound (i.e., uncomplexed) and complexed forms of the protein as it exists in its natural and/or isolated state. The term “protein” includes a protein subunit (i.e., a single polymer) that is part of a multisubunit protein.

As used herein, the terms “multisubunit protein complex” and “protein complex” are used interchangeably and refer to a protein that comprises complexes (i.e., aggregates) of two or more protein subunit components that are liked together by non-covalent bonds. Such proteins are also referred to as “multimeric” proteins or “oligomers,” with binary (two subunits), ternary (three subunits) and quaternary (four subunits) being the most common forms. Examples of multimeric proteins include, but are not limited to, troponin I, myoglobin, hemoglobin, alpha keratin, collagen, cytochrome C, lysozyme, ribonuclease, chymotrypsin, papain, carboxypeptidase A, and gamma globulin.

The term “unbound form” refers to a single amino acid polymer, such as a protein subunit component of a multimeric protein complex, that is not associated or complexed with one or more protein subunits or with another molecule or substance. For example, the individual cTnI protein subunit is considered to be an unbound protein. Similarly, the phosphorylated, oxidized, and proteolytically degraded forms, as well as the various isoforms, of individual proteins (such as cTnI) are considered to be “unbound forms” of the protein.

An “isoform” is a protein having the same function and similar (or identical sequence) as another protein but which is the product of a different gene, and usually is tissue specific.

As used herein, the term “complexed form” refers to a protein (e.g., a protein subunit) which is bound to other proteins (e.g., other protein subunits) to form a protein complex. Examples of protein complexes include binary, ternary, and quaternary protein complexes. The term “complexed form” also includes a protein unit that is bound to a molecule or other entity other than another protein unit.

The term “total protein” or “total protein concentration” refers to the total amount of a protein of interest in a sample, that is, the total amount of both the unbound and complexed forms of the protein in the sample. For example, the total concentration of cTnI in a sample includes the total amount of unbound cTnI as well as cTnI in its oxidized form, phosphorylated form, binary forms of cTnI such as I/T and I/C, and the ternary troponin complex I/T/C.

As used herein, the term “factor” refers to a chemical, biochemical, or other compound or substance that alters the conformation of the various forms of a protein to expose or facilitate the binding of a specific epitope of the protein to its binding partner. Factors utilized in the method of this invention do not include factors which result in complete dissociation of multisubunit protein complexes into individual uncomplexed subunits. It is known that various biochemical parameters (such as ATPase, protein kinase, phosphatase, oxidation of —SH group, etc.) are implicated in the highly complex mechanism of protein subunit interactions in multisubunit protein complexes such as troponin. It is a discovery of this invention that when these parameters are properly introduced into the sample or assay reaction mixture by the addition of an appropriate factor, conformational changes of the various forms of the protein will occur, which in turn impact the exposure and affinity of the epitope site for its binding partner. As a result, the binding partner will have equimolar access to the specific epitope on all forms of the protein. When used in an assay according to the methods of this invention, the factor can be added directly to the test sample or it can be added to the reaction mixture comprising the sample and the reagent particles.

The terms “equimolar access” or “equimolar recognition” are used interchangeably and refer to the ability of binding partner to equally access and bind to its epitope on all forms of a protein of interest in a sample.

As used herein, the term “equimolar measurement” refers to the equivalent measurement of the concentration of all free and complexed forms of a protein of interest in a sample.

The term “epitope” refers to the site on protein or antibody that binds to a binding partner specific for the analyte.

The term “binding partner” refers to a molecule or substance that specifically recognizes and binds to an analyte of interest (e.g., a protein), and exhibits negligible cross-reactivity with other molecules or substances. Typical binding partners include, but are not limited to, polyclonal antibodies, monoclonal antibodies, antibody fragments, antigens, antigen fragments, receptors, and nucleic acids. Such binding partners specific for a given analyte may be obtained from commercial sources or may be prepared in accordance with standard procedures known to those skilled in the art. The binding partner may be labeled with a reporter group using methods well known to those skilled in the art.

As used herein, the term “reagent particle” refers to an insoluble particle having at least a first coating immobilized on its surface. When used in direct particle-enhanced assays, the reagent particle may be a single-coated particle. The coating comprises a binding partner for the analyte of interest in a test sample. When used in competitive particle-enhanced assays, the coating may be the analyte of interest or an analog of the analyte. The coating can be attached directly to the particle by covalent (chemical) bonds or non-covalent bonds (e.g., physical adsorption). Alternatively, the coating can be indirectly attached via a linkage to the particle, for example, through a biotin/avidin linkage. Biotin/avidin linkages and other such linkages for immobilizing a binding partner or analyte to a carrier particle are well known to those skilled in the art. The term “reagent particle” further includes the improved reagent particles of this invention comprising insoluble particles having two or more coatings. One example of such particles are double-coated particles of this invention having an inner coating immobilized on the surface of the particle and an outer coating comprising a binding partner (e.g., an antibody) specific for the analyte of interest immobilized on the inner coating, as discussed below in detail.

Any suitable insoluble particle known in the art for particle-enhanced immunoassays may be employed for purposes of this invention. Examples of suitable particles include, but are not limited to, polystyrene particles (commonly referred to as latex particles), glass, acrylamide, methacrylate, nylon, acrylonitrile, polybutadiene, metals, metal oxides and their derivatives, dextran, cellulose, and liposomes, and natural particles such as red blood cells, pollens, and bacteria. The insoluble particle can be of any suitable size.

As discussed above, one embodiment of this invention improves conventional assays for the detection of proteins by changing the reaction environment of the protein. The changes in the reaction environment result in conformational changes in the unbound and/or complexed forms of the protein, such that the epitope on all forms of the protein is exposed. This in turn allows equal access of the binding partner to its specific epitope on all forms of the protein. Equal access to the epitope results in consistent binding between the epitope and binding partner and consistent aggregation of reagent particles, thereby increasing the accuracy of the measurement of total protein concentration in a sample. As a result, discordant patient results in protein assays will be reduced due to improvement of the accuracy in measuring total protein concentration in the sample.

The embodiments discussed below for improving equimolar detection by changing the reaction environment of a protein of interest will be discussed using the troponin subunit cTnI as an example of a protein of interest. However, it is to be understood that the following embodiments are used merely to illustrate the basic concepts presented in this disclosure and are not meant to be limiting in any way.

One embodiment of this invention for assaying a protein of interest in a sample comprises modifying the reaction environment of the protein by adding to the sample or the assay reaction mixture a factor that alters the activity of the ATPase. ATPase is an enzyme that plays an important role in the activity and structure of both unbound and complexed proteins. Examples of compounds that alter the activity of ATPase include, but are not limited to, ATPase substrates, ATPase inhibitors, and metal cofactors such as magnesium (Mg) and zinc (Zn). An ATPase substrate is a compound upon which ATPase will exert catalytic activity, or which causes reactions that lead to the liberation of inorganic phosphorus from ATP. Examples of ATPase substrates include, but are not limited to, adenosine triphosphate (ATP) analogs, guanosine triphosphate (GTP), and magnesium adenosine triphosphate (MgATP₂). Examples of ATPase inhibitors include, but are not limited to, vanadate, 4-chloro-7-nitrobenzofuran, dicyclohexylcarbodiimide, sodium fluoride (NaF), sodium azide (NaN₃), caffeine, GTP, and uridine 5′-diphosphate glucose (UDP-glucose). When ATPase activity is altered by the above factors, the ATPase inhibiting effect of cTnI is also accordingly altered, thereby eliciting conformational changes in cTnI.

Another embodiment of an assay of this invention comprises modifying the reaction environment of a protein by adding a factor that modifies the phosphorylation state of the protein of interest. For example, the conformation of the protein subunit cTnI is reported to be affected by phosphorylation/dephosphorylation (Biophys. J., 63:986-995 (1992); Biochem., 33:12729-12734 (1994)). A compound that modifies the phosphorylation state of a protein includes any natural or synthetic compound that inhibits or enhances (i.e., modifies) the activity or function of protein kinase or protein phosphatase. Depending on the concentration of the factor, the same modifier can act either as an enhancer or an inhibitor of protein kinase activity. Similarly, depending on the concentration, a protein phosphatase modifier can act either as an enhancer or as an inhibitor of protein phosphatase activity. An “enhancer” is a compound that increases, augments, or facilitates the activity of another compound. Examples of protein kinase modifiers include, but are not limited to, tacrolimis (also known as FK 506), indole carbazoles, N₆-dimethylaminopurine, olomoucine, rapamycin, and synthetic peptides for protein kinase. Examples of protein phosphatase modifiers include, but are not limited to, akadaic acid, microcystine R, and phosphatase Inhibitor 2.

Another embodiment of this invention comprises modifying the reaction environment of a protein by adding a factor that modifies the reactivity of thiol groups of a protein of interest. For example, the protein subunit Troponin I contains two of the thiol-containing amino acid cysteine, one at position 80 and another at position 97 (FEBS Letters, 270:57-61 (1990)). During the purification of troponin I from tissues, the oxidation state of troponin I is directed toward the reduced form using various reductants, including mercaptoethanol, dithiothreitol and the like (Can. J. Biochem., 54:546-554 (1976); Methods Enzymol., 85:241-263 (1982)). After purification, the art teaches to maintain troponin I in the reduced form to prevent intermolecular disulfide formation (i J. Biol. Chem., 258:2951-2954 (1983)). It has been reported (U.S. Pat. No. 6,174,686) that troponin I can rapidly oxidize intramolecularly which alters the conformation of the protein. The apparent instability in the troponin I molecule has been attributed to the dynamics of the intramolecular oxidation or reduction of the troponin I molecule.

Any compound that modifies the reactivity of thiol groups is suitable for purposes of this invention. One example of a factor that modifies the reactivity of thiol groups is a thiol-reducing agent, wherein the reducing agent prevents conformational changes in the protein by preventing intramolecular disulfide formation. Examples of a thiol-reducing agent which maintain the protein in the reduced form include, but are not limited to, dithioerythritol (DTE) and dithiothreitol (DTT). Another example of a factor that modifies the reactivity of thiol groups is an antioxidant that maintains the protein in the reduced form. Suitable antioxidant factors which may be used in this invention included, but are not limited to, ascorbic acid and tocopherol.

Another embodiment of this invention comprises modifying the reaction environment of a protein by adding a calcium chelator which alters the conformation of the unbound and/or complexed forms of the protein. Examples of calcium chelators include ethylenediaminetetraacetic acid (EDTA) and its salts, and etbylenebis(oxyethylenenitrilo)tetraacetic acid (EGTA).

As will be appreciated by those of ordinary skill in the art, the method of adding the above-described factors that improve equimolar recognition of epitopes may be applied to improve any type of assay. Such immunoassays include, but are not limited to, particle-enhanced immunoassays, ELISA, LIA (luminescence immunoassay), FIA (fluorescence immunoassay), and CIA (chemiluminescence assay). Further, the concentration of total protein subunit may be determined by various methods known to those skilled in the art, including, but not limited to, light-scattering methods such as turbidimetry nephelometry.

The methods of this invention can be used to detect a protein of interest in any number of samples. As used herein, the term “sample” includes, but is not limited to, biological samples such as blood, plasma, serum, saliva, cerebral spinal fluid, urine, and any other type of fluid, cell, tissue, or material which may contain an analyte of interest. By “cell,” it is meant any sample obtained from a bodily organ, embryo, or other tissue, e.g., a tumor, from which a cellular suspension can be prepared. The sample may be diluted, undiluted, treated, or untreated.

This invention further provides an assay kit for determining the total amount of protein in a sample, wherein the sample may comprise a mixture of both unbound and complexed forms of the protein. The kit contains at least one container containing reagent particles having immobilized thereon a binding partner for the protein, and another container containing a factor which causes conformational changes in the various forms of the protein. The factor includes, but is not limited to, an ATPase substrate, an ATPase inhibitor, a metal cofactor, a protein kinase inhibitor, a protein kinase enhancer, a phosphatase inhibitor, a phosphatase enhancer, a thiol-reducing agent, an antioxidant and a calcium chelator.

Yet another aspect of this invention provides a method of improving the sensitivity of particle-enhanced assays by providing improved reagent particles for use in detecting an analyte of interest in a sample. More specifically, one aspect of this invention provides an improved reagent particle comprising two or more coatings. One example of an improved reagent particle is a double-coated insoluble particle, as illustrated in FIG. 2, comprising an inner and an outer coating. In the example shown in FIG. 2, the inner coating comprises a compound such as an antibody or a protein immobilized on the insoluble particle by covalent or non-covalent bonds. The outer coating comprises a binding partner that is specific for the analyte of interest, where the outer coating is immobilized on the inner coating. Examples of binding partners include, but are not limited to, monoclonal antibodies, polyclonal antibodies, antibody fragments, antigens, antigen fragments, receptors, and nucleic acids. As a result of the double coating, the steric orientation of the antibody of the outer coating is modified to a more desirable orientation relative to conventional reagent particles, thereby improving access of the antibody binding site to the analyte of interest.

As used herein, “immobilized” refers to the covalent or non-covalent attachment of an inner coating to the insoluble particle. The term “immobilized” also refers to the covalent or non-covalent attachment of one coating to another coating, such as the attachment of the outer coating to the inner coating, as illustrated in FIG. 2.

In one embodiment of this invention, as shown in FIG. 2, the outer coating is an antibody that is immunoreactive with the analyte of interest. As used herein, the term “antibody” refers in general to any molecule that is capable of binding to the epitopic determinant of an analyte. The antibody includes immunoglobulins, including intact molecules as well as functional fragments thereof, such as Fab, F(ab′)₂, and Fv.

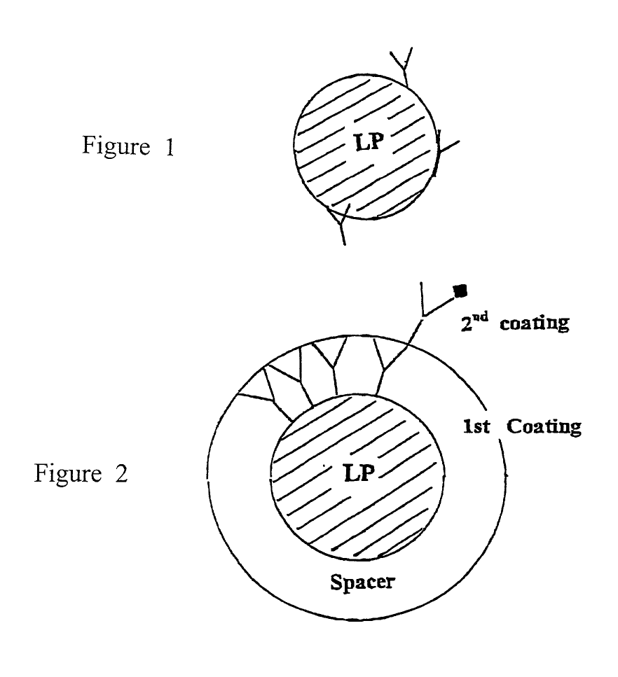

The simplest antibody molecules are Y-shaped molecules with two identical analyte binding sites (epitopes)—one at the tip of each arm of the Y. The arms of the “Y” are referred to as the “Fab” fragments, and the tail of the “Y” is referred to as the “Fc” fragment. One type of a conventional reagent particle for particle-enhanced assays comprises an insoluble particle having an antibody specific for the analyte of interest immobilized on its surface. FIG. 1 illustrates the random orientation of antibodies immobilized on a latex particle (LP) used in conventional particle-enhanced immunoassays. As a result of this random orientation, some of the particle-bound antibodies will be undesirably aligned on the latex particle, such that one (or both) of the specific binding sites of the antibody (i.e., a Fab fragment) is unable to bind to the analyte of interest. Such undesirable alignment reduces the specific activity of conventional latex-bound antibody, and consequently reduces the sensitivity of an assay utilizing these conventional particles.

Ideally, the Fe site of the antibody (i.e., the tail of the “Y”) is bound to the latex particle, thereby leaving the binding sites on the Fab fragments free to bind to the analyte. This desired alignment of the analyte-specific antibody on the particle is achieved in one embodiment of this invention with double-coated reagent particles, as illustrated in FIG. 2. Preferably, the inner coating comprises an antibody having an Fc fragment with a higher affinity than the Fab fragments for the insoluble particle. Further, the outer coating is preferably an antibody having an Fc fragment with a higher affinity than the Fab fragments for the Fab fragment of the inner antibody coating. The inner coating effectively serves as a “spacer” or an orientation guide for the outer coating. This allows the antibodies of the outer coating to spread out more so that the Fab fragments are oriented for easy access and binding to the analyte. The double coating thus results in enhanced recognition of the analyte-specific antibody by the analyte and increased sensitivity of the assay.

The inner coating may be a protein or a polyclonal or monoclonal antibody. It is not necessary that the inner coating be a binding partner for the analyte. Thus, the inner coating may be the same or a different antibody than the outer coating. In the example shown in FIG. 2, the inner coating is an antibody having the tail of the “Y” (the Fc fragment) bound to the particle (LP) by covalent or non-covalent bonds. As described above, any suitable particle known in the art for particle-enhanced immunoassays may be employed for purposes of this invention. In a preferred embodiment, the insoluble particles are polystyrene (i.e., latex) particles. The inner antibody coating can be immobilized on the particles in accordance with standard techniques well known to those skilled in the art, such as physical (passive) absorption, facilitated (forced) absorption, and covalent coupling. Alternatively, commercially available coated latex particles, such as protein G-coated latex particles, may be used to prepare the double-coated particles of this invention.

In the example illustrated in FIG. 2, the outer coating comprises an antibody that is immunoreactive with the analyte of interest. Preferably, the antibody is oriented, as shown in FIG. 2, having its “Y” tail bound to one of the arms (Fab fragment) of the inner coating. Preferably, the antibody of the outer coating is a monoclonal antibody; however, it may also be a polyclonal antibody. The outer antibody coating is immobilized on the inner antibody coating by covalent or non-covalent bonds in accordance with standard techniques well known to those skilled in the art.

While the example illustrated in FIG. 2 shows a latex particle having a double coating of antibodies, it will be appreciated that additional coatings may be immobilized on the reagent particle. Therefore, this invention also includes reagent particles having three, four, etc. coatings. Further, it will be understood by those skilled in the art that this invention includes improved reagent particles wherein the outer coating may be a binding partner other than an antibody, as described above.

The improved reagent particles of this invention may be used in any particle-enhanced assay for the detection of analytes, including direct and competitive assays. Thus, this invention further includes assays having increased sensitivity using the improved reagent particles. For example, the improved reagent particles may be used in particle-enhanced aggregation reactions in which aggregates of reagent particles and analyte are formed. The extent of aggregation indicates the presence and/or amount of analyte in the sample. The amount of analyte in the sample can be measured by methods known to those of ordinary skill in the art, such as various light-scattering methods, particle counting, nephelometry, and turbidimetry. Any instrument capable of measuring aggregation can be used to detect and/or measure the analyte in the sample. The improved reagent particles may further comprise a label or reporter group to aid the detection of aggregated particles. The reporter group can be part of the inner coating, the outer coating, or the insoluble particle. Suitable reporter groups are well known to those skilled in the art and need not be described further.

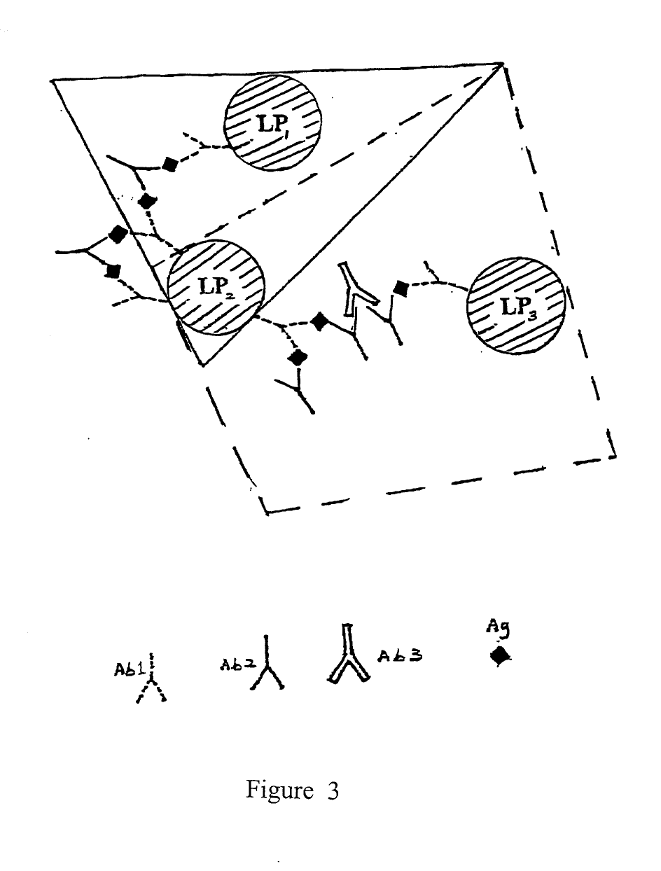

Yet another aspect of this invention provides a method of enhancing the sensitivity of particle-enhanced immunoassays by increasing the size of the particle aggregates that are formed in the immunoassay reaction mixture, thereby increasing the detection signal. A typical particle-enhanced immunoassay for determining the concentration of an analyte of interest in a sample involves first immobilizing an analyte-specific antibody on the insoluble particles, and mixing the particle-bound antibody with the sample under conditions that will allow aggregation of the particles to occur. In a direct particle-enhanced immunoassay, the presence of the target analyte is determined by the formation of any aggregates that form as a result of binding between the particle-bound antibody and the analyte. The extent of aggregation is proportional to the concentration of the analyte in the sample. The utility of a particle-enhanced assay in measuring the concentration of an analyte in a sample depends upon its capacity to measure the extent of the formation of the aggregates. If the aggregates are large enough, they will become capable of scattering light, or of spontaneously precipitating. In such cases, detection methods such as nephelometric, turbidimetric or other methods known to those skilled in the art are used for determining the extent of particle aggregation. However, such methods require the production of large aggregates, which may not be possible when the concentration of the analyte in the sample is very low (e.g., less than or equal to nanogram per milliliter levels), as shown in FIG. 3. In FIG. 3, the area inside the solid triangle illustrates the degree of particle aggregation in a sample containing very low concentrations of the analyte (Ag) once the sample solution has reach an equilibrium state. The extent of this aggregation can be enhanced to a higher level with the addition of a third (linker) antibody according to the method of this invention, which in turn results in a higher signal and an increased sensitivity of the assay.

Accordingly, another embodiment of this invention provides a method of enhancing the sensitivity of a particle-based immunoassay by increasing the extent of particle aggregation as well as increasing the size of the particle aggregates in the assay reaction mixture. More specifically, another embodiment of a particle-enhanced assay of this invention as illustrated in FIG. 3 comprises forming a reaction mixture by combining a sample containing an unknown amount of analyte with:

a) reagent particles comprising insoluble particles (LP) having immobilized thereon a first antibody (Ab₁) that is immunoreactive with a first epitope of the analyte (Ag);

b) a second antibody (Ab₂) that is immunoreactive with a second epitope of the analyte (Ag); and

c) a linker antibody (Ab₃) which specifically binds the second antibody (Ab₂) but does not bind to the analyte (Ag). As used herein, a “linker” antibody refers to an antibody that binds to an anti-analyte antibody but does not bind to the analyte.

The reaction mixture is incubated under conditions that allow binding between the immobilized first antibody (Ab₁) and the analyte (Ag), binding between the second antibody (Ab₂) and the analyte (Ag), and binding between the second antibody (Ab₂) and the linker antibody (Ab₃). Suitable incubation conditions used in particle-enhanced assays are well known to those skilled in the art and can be determined without undue experimentation. Preferably, the second antibody (Ab₂) binds an epitope that is different than the epitope to which the immobilized first antibody (Ab₁) binds.

According to a direct assay of this invention utilizing three antibodies as described above, the presence of the analyte (Ag) in the sample results in the formation of aggregates that are larger than those formed in assays which do not include the linker antibody (Ab₃). One example of a larger aggregate formed in an assay mixture that includes a linker antibody (Ab₃) is shown in the area inside the dashed square in FIG. 3 and can be represented by Equation 1.

[LP₂/Ab₁ [symbol] Ag [symbol] Ab₂] [symbol] Ab₃ [symbol] [Ab₂ [symbol] Ag [symbol] Ab₁/LP₃] (1)

The formation of these larger aggregates produces a higher detection signal for the assay. As a result, the sensitivity of the assay is increased.

The particle bound (immobilized) first antibody (Ab₁) is an antibody specific for the analyte. The immobilized antibody may be a monoclonal or polyclonal antibody or an antibody fragment, and can be naturally occurring or synthetically prepared according to standard methods. The first antibody can be immobilized on the insoluble particle by covalent or non-covalent bonds and in accordance with standard techniques well known to those skilled in the art, such as physical (passive) absorption, facilitated (forced) absorption, and covalent coupling.

The second antibody (Ab₂) is different than the first antibody but is also specific for the analyte (Ag) of interest. Preferably the second antibody binds to an epitope on the analyte that is different than an epitope that binds the first antibody. The second antibody may be a monoclonal or polyclonal antibody or an antibody fragment, and can be naturally occurring or synthetically prepared according to standard methods. The second antibody can be free (i.e., not immobilized on a particle) as shown in FIG. 3, or, alternatively, the second antibody can be immobilized on an insoluble particle (not shown).

The linker antibody (Ab₃) is an antibody that specifically binds the nonimmobilized second antibody (Ab₂) but does not bind the analyte (Ag). The linker antibody (Ab₃) is not immobilized on an insoluble particle. Rather, the purpose of the linker antibody (Ab₃) is to enhance aggregation of the particle (LP) by binding or linking two of the second antibodies (Ab₂), as shown in FIG. 3. The linker antibody (Ab₃) can be prepared according to methods known by those skilled in the art. The linker antibody (Ab₃) in general is polyclonal and can be naturally occurring or synthetic.

The above-described assay utilizing three different antibodies may be used in any type of particle-enhanced immunoassay for the detection of analytes. As used herein, an “analyte” refers to the substance whose presence and/or concentration in a sample is to be determined. The term “analyte” refers to any substance for which there exists a specific antibody, or for which an antibody can be prepared. Representative analytes include, but are not limited to, proteins, subunits of multisubunit proteins, antigens, haptens, antibodies, proteins, peptides, amino acids, hormones, steroids, cancer cell markers, tissue cells, viruses, vitamins, drugs, nucleic acids, pesticides, receptors, and metabolites.

The amount of analyte in the sample can be measured by any method capable of measuring the aggregation of particles, such as various light-scattering methods, particle counting, nephelometry, and turbidimetry. Any instrument capable of measuring the aggregation of particles, such as the SYNCHRON LX-PRO, IMMAGE and ACCESS instruments from Beckman Coulter can be used to detect and/or measure the analyte in the sample. Alternatively, the particle (LP) or any of the antibodies, Ab₁, Ab₂ or Ab₃, can be labeled for detection. Suitable labels include enzymes, fluorophores, chromophores, radioisotopes, dyes, colloidal gold, and chemiluminescent agents.

In another embodiment of this invention utilizing three different antibodies, the sensitivity of the immunoassay is increased by varying the order in which the components of the assay are combined. For example, typically an assay of this invention can be performed by adding the Ab₁/LP reagent particle to the sample, followed by the addition of the second (Ab₂) antibody and linker antibody (Ab₃). However, depending on the design of the immunoassay and the different binding affinities between the analyte and the immobilized antibody Ab₁/LP and the binding affinities of the antibodies for each other, the order of the addition of the assay components can be designed and controlled to enhance the binding of each component. The end result will be enhanced particle aggregation in the assay reaction mixture and enhanced signal detection.

For example, if the binding affinity between the analyte and the second antibody is much higher than that between the second antibody and the linker antibody, the addition of the second antibody and the linker antibody to the reagent particles in an assay medium can be made prior to the addition of the sample suspected of containing the analyte (Ag).

In an example of a competitive (inhibition) type of immunoassay (not shown) where the sample analyte (Ag) and an immobilized analyte (LP/Ag) compete for binding with the immobilized antibody (LP/Ab), typically the sample analyte (Ag) is introduced simultaneously with the immobilized analyte (LP/Ag) to an assay medium containing a limited amount of an analyte-specific immobilized antibody (LP/Ab), resulting in equal competition between the analyte and the immobilized analyte (LP/Ag) for the immobilized antibody (LP/Ab). However, if the binding affinity between the sample analyte and the immobilized antibody (LP/Ab) is much weaker than the binding between the immobilized analyte (LP/Ag) and the immobilized antibody (LP/Ab), then the adding sequence can be altered such that the sample analyte is first combined with the antibody for a period of time, followed by the addition of the immobilized analyte (LP/Ag). This change in the adding sequence will offer equal competition between the sample analyte and the immobilized analyte (LP/Ag) for the antibody, especially in the presence of a very low concentration of sample analyte, and thus will enhance the reverse signal, since in a competitive assay, the extent of aggregation is indirectly proportional to the concentration of analyte in the sample. In addition, this method results in a much more sensitive detection of low concentrations of analyte.

The assays described herein improve the sensitivity and the low detection limits of high value analytes such as cTnI. For example, the assays further improve the specific binding and epitope recognition between the analyte of interest and the antibody. In addition, the assays provide equimolar measurements of all forms of a protein in a sample, thereby minimizing discordant measurements of the analytes in test samples among different assay methods.

### EXAMPLES

### Example 1

Modifying the Reaction Environment with Metal Cofactors

Two monoclonal antibodies (HTI M6 and clone 284) conjugated to 537 nm latex particles (blocking with glycine) were used as the reagent particles in a direct sandwich binding particle-enhanced immunoassay. The particles were added to the sample containing very low concentrations of sample analyte cardiac Troponin I/C/T (Scipac, Sittingbourne, UK) in a base buffer (Tris-NaCl, choline, BSA, and 0.4% PVA, pH 7.5). Either magnesium chloride (MgCl₂) or zinc chloride (ZnCl₂) was added to the reaction mixture.

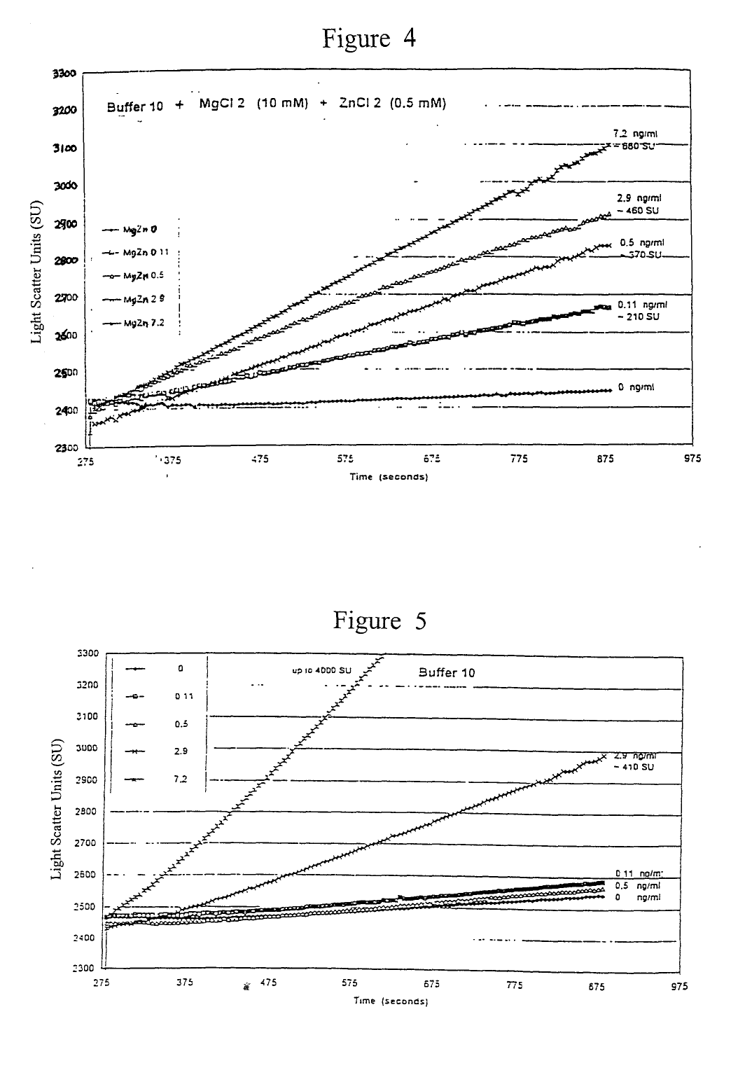

The graphs shown in FIGS. 4 and 5 summarize the results of this experiment. FIG. 4 is a graph showing the detection and separation of different levels of cTnI without the addition of a MgCl₂ or ZnCl₂ to the assay reaction mixture. Without the addition of a metal cofactor, the signal for the low concentrations of analyte (0, 0.5, and 0.11 ng/mL) is indistinguishable. FIG. 5 is a graph showing the detection and separation of different levels of cTnI after the addition of a metal cofactor (MgCl₂ or ZnCl₂) to the assay reaction mixture. With the addition of the metal cofactors to the reaction mixture, lower concentrations of the analyte (0, 0.5, and 0.11 ng/mL) were detected, and the resolution among the low levels of analyte was improved.

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow.

We claim:

1. An assay for determining the amount of total protein in a sample, comprising:
a) providing a sample containing an unknown amount of the protein, wherein the sample may comprise both unbound and complexed forms of the protein;
b) providing reagent particles comprising insoluble particles having immobilized thereon a binding partner specific for an epitope of the protein;
c) forming a reaction mixture by combining said sample, said reagent particles, and a factor, wherein the factor causes conformational changes in the unbound and/or complexed forms of the protein to allow the epitope on all forms of the protein to be accessible to said binding partner;
d) incubating said reaction mixture under conditions that allow binding between the immobilized binding partner and the epitope on all forms of the protein, wherein said binding results in aggregation of said particles; and
e) determining the amount of total protein in the sample.

2. The assay of claim 1, wherein said factor comprises a compound that alters ATPase activity.

3. The assay of claim 2 wherein said factor is an ATPase substrate, an ATPase inhibitor, or a metal cofactor.

4. The assay of claim 3, wherein said ATPase substrate is selected from the group consisting of ATP analogs, GTP, and MgATP₂.

5. The assay of claim 3, wherein said ATPase inhibitor is selected from the group consisting of vanadate, 4-chloro-7-nitrobenzofuran, dicyclohexylcarbodiimide, NaF, NaN₃, caffeine, GTP, and UDP-glucose.

6. The assay of claim 3, wherein said metal cofactor is selected from the group consisting of magnesium and zinc.

7. The assay of claim 1, wherein said factor is a protein kinase modifier.

8. The assay of claim 7, wherein said protein kinase modifier is selected from the group consisting of tacrolimis, indole carbazoles, N₆-dimethylaminopurine, olomoucine, rapamycin, and synthetic peptides for protein kinase.

9. The assay of claim 1, wherein said factor is a protein phosphatase modifier.

10. The assay of claim 9, wherein said protein phosphatase modifier is selected from the group consisting of akadaic acid, microcystine R, and phosphatase Inhibitor 2.

11. The assay of claim 1, wherein said factor is a thiol-reducing agent.

12. The assay of claim 11, wherein said thiol-reducing agent is selected from the group consisting of dithiothreitol and dithioerythritol.

13. The assay of claim 11, wherein said thiol-reducing agent prevents the formation of an intramolecular disulfide bridge in said protein.

14. The assay of claim 1, wherein said factor is an antioxidant.

15. The assay of claim 14, wherein said antioxidant is selected from the group consisting of ascorbic acid and tocopherol.

16. The assay of claim 1, wherein said protein is cardiac troponin I.

17. The assay of claim 1, wherein said binding partner is selected from the group consisting of polyclonal antibodies, monoclonal antibodies, antibody fragments, antigens, antigen fragments, receptors and nucleic acids.

18. The assay of claim 1, wherein said reagent particle further comprises an inner coating immobilized on said insoluble particle, wherein said binding partner is immobilized on said inner coating.

19. The assay of claim 18, wherein said inner coating is selected from the group consisting of proteins, a monoclonal antibodies, polyclonal antibodies, and antibody fragments.

20. The assay of claim 1, further comprising adding to said sample a second antibody specific for a second epitope of said protein and a linker antibody which specifically binds said second antibody but does not bind to said protein.

21. The assay of claim 20, wherein said second antibody is immobilized on an insoluble particle.

22. The assay of claim 1, wherein the extent of said aggregation is indicative of the amount of said protein, wherein the extent of said aggregation is determined by nephelometry, turbidimetry, flow cytometry, or particle counting.

23. The assay of claim 1, wherein said binding partner is immobilized on said insoluble particles by covalent or non-covalent bonds.

24. The assay of claim 1, wherein said insoluble particles are selected from the group consisting of latex, glass, acrylamide, methacrylate, nylon, acrylonitrile, polybutadiene, metals, metal oxides, dextran, cellulose, liposomes, red blood cells, pollens, and bacteria.

25. An assay for determining the amount of analyte in a sample, comprising:
a) providing a sample containing an unknown amount of said analyte;
b) providing reagent particles having two or more coatings, comprising:
an insoluble particle;
an inner coating immobilized on said insoluble particle; and
an outer coating comprising a binding partner that specifically binds said analyte;
c) forming a reaction mixture by combining said reagent particles and said sample in an assay buffer;
d) incubating said reaction mixture under conditions that allow binding between said binding partner and said analyte, wherein said binding causes aggregation of said particles; and
e) determining the amount of analyte in said sample.

26. The assay of claim 25, wherein said inner coating is immobilized on said insoluble particles by covalent or non-covalent bonds.

27. The assay of claim 25, wherein said inner coating is selected from the group consisting of proteins, monoclonal antibodies, polyclonal antibodies, and antibody fragments.

28. The assay of claim 25, wherein said binding partner is selected from the group consisting of polyclonal antibodies, monoclonal antibodies, antibody fragments, antigens, antigen fragments, receptors, and nucleic acids.

29. The assay of claim 25, wherein when said reagent particles have two coatings, said outer coating is immobilized on said first coating by covalent or non-covalent bonds.

30. The assay of claim 25, wherein said insoluble particles are selected from the group consisting of latex, glass, acrylamide, methacrylate, nylon, acrylonitrile, polybutadiene, metals, metal oxides, dextran, cellulose, liposomes, red blood cells, pollens, and bacteria.

31. The assay of claim 25, wherein said analyte is a protein and said sample may comprise a mixture of unbound and complexed forms of said protein, and wherein said binding partner binds a specific epitope of said protein.

32. The assay of claim 31, further comprising adding a factor to said sample, wherein said factor causes conformational changes in said unbound and/or complexed forms of said protein to allow said epitope on all forms of said protein to be accessible to said binding partner.

33. The assay of claim 32, wherein said factor is selected from the group consisting of an ATPase substrate, an ATPase inhibitor, a metal cofactor, a protein kinase modifier, a phosphatase modifier, a thiol-reducing agent, an antioxidant, and calcium chelator.

34. The assay of claim 33, wherein said factor is selected from the group consisting of ATP analogs, GTP, MgATP₂, vanadate, 4-chloro-7-nitrobenzofuran, dicyclohexylcarbodiimide, NaF, NaN₃, caffeine, GTP, UDP-glucose, magnesium, zinc, tacrolimis, indole carbazoles, N₆-dimethylaminopurine, olomoucine, rapamycin, synthetic peptides for protein kinase, akadaic acid, microcystine R, phosphatase Inhibitor 2, dithiothreitol, dithioerythritol, ascorbic acid, tocopherol, ethylenediaminetetraacetic acid, and ethylenebis(oxyethylenenitrilo)tetraacetic acid.

35. The assay of claim 25, wherein said analyte is cardiac troponin I.

36. The assay of claim 25, wherein the extent of said aggregation is indicative of the amount of said analyte in said sample, wherein the extent of said aggregation is determined by nephelometry, turbidimetry, flow cytometry, or particle counting.

37. An assay for determining the amount of total protein subunit of a multisubunit protein in a sample, the assay comprising:
a) providing a sample containing an unknown amount of said protein subunit, wherein said sample may comprise both unbound and complexed forms of said protein subunit;
b) providing reagent particles having two or more coatings, comprising:
an insoluble particle;
an inner coating immobilized on said insoluble particle; and
an outer coating comprising a binding partner that binds a specific epitope of said subunit;
c) providing a factor, wherein said factor causes conformational changes in said unbound and/or complexed forms of said protein subunit to allow said epitope of all forms of said protein subunit to be accessible to said binding partner;
d) forming a reaction mixture by combining said sample, said particles, and said factor;
e) incubating said reaction mixture under conditions that allow binding between said binding partner and said epitope, wherein said binding causes aggregation of said particles; and
f) determining the amount of said protein subunit in said sample.

38. The assay of claim 37, wherein said factor is selected from the group consisting of an ATPase substrate, an ATPase inhibitor, a metal cofactor, a protein kinase modifier, a protein phosphatase modifier, a thiol-reducing agent, an antioxidant, and calcium chelator.

39. The assay of claim 38, wherein said factor is selected from the group consisting of ATP analogs, GTP, MgATP₂, vanadate, 4-chloro-7-nitrobenzofuran, dicyclohexylcarbodiimide, NaF, NaN₃, caffeine, GTP, UDP-glucose, magnesium, zinc, tacrolimis, indole carbazoles, N₆-dimethylaminopurine, olomoucine, rapamycin, synthetic peptides for protein kinase, akadaic acid, microcystine R, phosphatase Inhibitor 2, dithiothreitol, dithioerythritol, ascorbic acid, tocopherol, ethylenediaminetetraacetic acid, and ethylenebis(oxyethylenenitrilo)tetraacetic acid.

40. The assay of claim 37, wherein said inner coating is immobilized on said insoluble particles by covalent or non-covalent bonds.

41. The assay of claim 37, wherein said inner coating is selected from the group consisting of monoclonal antibodies, polyclonal antibodies, antibody fragments, and proteins.

42. The assay of claim 37, wherein when said reagent particles comprise two coatings, said outer coating is immobilized on said inner coating by covalent or non-covalent bonds.

43. The assay of claim 37, wherein said binding partner is selected from the group consisting of polyclonal antibodies, monoclonal antibodies, antibody fragments, antigens, antigen fragments, receptors, and nucleic acids.

44. The assay of claim 37, wherein said insoluble particles are selected from the group consisting of latex, glass, acrylamide, methacrylate, nylon, acrylonitrile, polybutadiene, metals, metal oxides, dextran, cellulose, liposomes, red blood cells, pollens, and bacteria.

45. The assay of claim 37, wherein the extent of particle aggregation is indicative of amount of total protein subunit in the sample, wherein the extent of aggregation is determined by nephelometry, turbidimetry, flow cytometry, or particle counting.

46. An assay for determining the amount of an analyte in a sample, the assay comprising:
a) forming a reaction mixture by combining in an assay medium:
i) a sample containing an unknown amount of said analyte;
ii) reagent particles comprising insoluble particles having immobilized thereon a first antibody that is immunoreactive with a first epitope of said analyte;
iii) a second antibody that is immunoreactive with a second epitope of said analyte, and
iv) a linker antibody which specifically binds said second antibody but does not bind to said analyte;
b) incubating said reaction mixture under conditions that allow binding between said immobilized first antibody and said analyte, between said second antibody and said analyte, and between said second antibody and said linker antibody, wherein said bindings cause aggregation of said particles; and
c) determining the amount of said analyte in said sample.

47. The assay of claim 46, wherein said analyte is a protein subunit of a multisubunit protein, wherein said sample may comprise both unbound and complexed forms of said protein subunit.

48. The assay of claim 47, further comprising adding to said sample a factor which causes conformational changes in said unbound and/or said complexed forms of said protein subunit to allow said epitope on all forms of said protein subunit to be exposed.

49. The assay of claim 48, wherein said factor is selected from the group consisting of an ATPase substrate, an ATPase inhibitor, a metal cofactor, a protein kinase modifier, a protein phosphatase modifier, a thiol-reducing agent, an antioxidant, and calcium chelator.

50. The assay of claim 49, wherein said factor is selected from the group consisting of ATP analogs, GTP, MgATP₂, vanadate, 4-chloro-7-nitrobenzofuran, dicyclohexylcarbodiimide, NaF, NaN₃, caffeine, GTP, UDP-glucose, magnesium, zinc, tacrolimis, indole carbazoles, N₆-dimethylaminopurine, olomoucine, rapamycin, synthetic peptides for protein kinase, akadaic acid, microcystine R, phosphatase Inhibitor 2, dithiothreitol, dithioerythritol, ascorbic acid, tocopherol, ethylenediaminetetraacetic acid, and ethylenebis(oxyethylenenitrilo)tetraacetic acid.

51. The assay of claim 46, wherein the extent of particle aggregation is indicative of the amount of analyte in the sample, wherein the extent of aggregation is determined by nephelometry, turbidimetry, flow cytometry, or particle counting.

52. The assay of claim 46, wherein said second antibody is immobilized on an insoluble particle.

53. The assay of claim 46, wherein step (a) further comprises first combining said reagent particles, said second antibody and said linker antibody to form a mixture, and then adding said sample to said mixture.

54. The assay of claim 46, wherein step (a) further comprises first combining said sample and said reagent particles to form a mixture, and then adding said second antibody and said linker antibody to said mixture.

55. An improved reagent particle comprising two or more coatings for detecting the presence of an analyte of interest in a particle-based immunoassay, said reagent particle comprising:
an insoluble particle;
an inner coating immobilized on said insoluble particle; and
an outer coating comprising a binding partner that specifically binds with said analyte of interest, wherein when said reagent particle comprises two coatings, said binding partner is immobilized on said inner coating.

56. The reagent particle of claim 55, wherein said particles are selected from the group consisting of latex, glass, acrylamide, methacrylate, nylon, acrylonitrile, polybutadiene, metals, metal oxides, dextran, cellulose, liposomes, red blood cells, pollens, and bacteria.

57. The reagent particle of claim 55, wherein said inner coating is immobilized on said insoluble particles by covalent or non-covalent bonds.

58. The assay of claim 55, wherein said inner coating is selected from the group consisting of monoclonal antibodies, polyclonal antibodies, antibody fragments, and proteins.

59. The assay of claim 55, wherein said binding partner is selected from the group consisting of polyclonal antibodies, monoclonal antibodies, antibody fragments, antigens, antigen fragments, receptors, and nucleic acids.

60. The reagent particle of claim 55, wherein said binding partner is immobilized on said inner coating by covalent or non-covalent bonds.

61. The reagent particle of claim 55, wherein said analyte is a protein subunit of a multisubunit protein complex.

62. A kit for determining the total amount of a protein in a sample, wherein said sample may comprise both unbound and complexed forms of said protein, comprising:
reagent particles having immobilized thereon a binding partner specific for an epitope of said protein; and
a factor which causes conformational changes in said unbound and/or complexed forms of said protein to allow said epitope of the free and complexed forms of said protein to be exposed.

63. The kit of claim 62, wherein said factor is selected from the group consisting of an ATPase substrate, an ATPase inhibitor, a metal cofactor, a protein kinase modifier, a phosphatase modifier, a thiol-reducing agent, an antioxidant, and calcium chelator.

64. The kit of claim 63, wherein said factor is selected from the group consisting of ATP analogs, GTP, MgATP₂, vanadate, 4-chloro-7-nitrobenzofuran, dicyclohexylcarbodiimide, NaF, NaN₃, caffeine, GTP, UDP-glucose, magnesium, zinc, tacrolimis, indole carbazoles, N₆-dimethylaminopurine, olomoucine, rapamycin, synthetic peptides for protein kinase, akadaic acid, microcystine R, phosphatase Inhibitor 2, dithiothreitol, dithioerythritol, ascorbic acid, tocopherol, ethylenediaminetetraacetic acid, and ethylenebis(oxyethylenenitrilo)tetraacetic acid.

65. The kit of claim 62, wherein said protein is a subunit of a multisubunit protein.

66. The kit of claim 62, wherein said reagent particles further comprise an inner coating immobilized on said particle, wherein said binding partner is immobilized on said inner coating.
